(19) United States
(12) Patent Application Publication
(10) Pub. No.: US 2017/0157802 A1
(43) Pub. Date: Jun. 8, 2017

(54) Sacrificial Templates Comprising a Hydrogel Cross-linking Agent and Their Use for Customization of Hydrogel Architecture

(71) Applicants: Randolph Scott Ashton, Madison, WI (US); Jason David McNulty, Stillwater, MN (US); Carlos Ruben Marti-Figueroa, Madison, WI (US); Lih-Sheng Turng, Madison, WI (US)

(72) Inventors: Randolph Scott Ashton, Madison, WI (US); Jason David McNulty, Stillwater, MN (US); Carlos Ruben Marti-Figueroa, Madison, WI (US); Lih-Sheng Turng, Madison, WI (US)

(21) Appl. No.: 14/961,033

(22) Filed: Dec. 7, 2015

(51) Int. Cl. B29C 33/52; A61L 27/52; C08J 3/075; C08J 9/26; B29C 33/38; C08J 9/00; A61L 27/56; A61L 27/58; B29C 39/02; B29C 33/44; A61L 27/20; C08J 3/24

(52) U.S. Cl. CPC B29C 33/52; A61L 27/20; A61L 27/52; C08J 3/075; C08J 9/26; C08J 3/24; C08J 9/0061; A61L 27/56; A61L 27/58; B29C 39/02; B29C 33/448; B29C 33/3842; C08J 2305/04; C08J 2201/026; C08J 2201/046; C08J 2207/10; C08J 2429/04; B29K 2105/0061

(57) ABSTRACT

Described herein are sacrificial templates generated from water soluble thermoplastic-divalent cation-composite materials, such as poly(vinyl alcohol)-calcium. Also described herein are methods for the use of such sacrificial templates in casting of precise internal space microarchitectures within hydrogels, such as microchannel networks within alginate hydrogels.

### BACKGROUND

Hydrogel biomaterials are used throughout the field of tissue engineering as versatile scaffolds to support 3D cell growth and shape the morphology of tissue constructs. Normal tissue development and physiology rely on proper cytoarchitectural organization at multiple length scales. Hence, several methods have been developed for engineering the macro-to-microscale architecture of hydrogel scaffolds including layer-by-layer 3D printing technologies, such as fused deposition modeling (FDM), and stereolithography (SLA). For example, FDM has been used to create interconnected 3D lattices composed of water-soluble carbohydrate glass filaments. Subsequently, the lattices can be encapsulated within hydrogels, and upon dissolution, leave behind channel networks suitable for generating microvasculature within prospective 3D tissues. Alternatively, sacrificial poly(vinyl alcohol), alginate, gelatin and PEG templates can be casted within SLA fabricated molds and similary used to engineer microscale hydrogel architecture.

While these approaches enable rapid casting of complex hydrogel architectures, the fabrication techniques and sacrificial template materials impose several limitations. First, the FDM and SLA/solvent casting fabrication techniques are not scalable for mass production due to extended manufacturing cycle times per sacrificial template. Second, carbohydrate glass templates are brittle and inelastic, and therefore have low durability. Third, current template materials have only been proven to effectively cast complex geometries within rapidly bulk curing hydrogels such as PEG, fibrin and methacrylated gelatin, which are suboptimal for some biological applications. Poorer dimensional accuracy is observed when using carbohydrate glass lattices to cast channels within diffusion limited, ionically crosslinked alginate hydrogels, a widely used tissue engineering scaffold and clinically approved biomaterial. Alginate in particular has several advantages for tissue engineering applications: facile and gentle cell encapsulation, ease of chemical modification via densely presented carboxylic acid groups on its polymer backbone, and enzymatic degradation mechanisms that are orthogonal to the mammalian genome. Thus, there is an ongoing need to develop a scalable mass production process for generating sacrificial templates that are mechanically resilient and capable of accurately casting architectural features within both bulk curing and diffusion limited, ionically crosslinked hydrogels.

### BRIEF SUMMARY

Disclosed herein are sacrificial templates comprising a water-soluble thermoplastic-divalent cation composite material (e.g., a poly(vinyl alcohol)-calcium salt composite material), methods for making them, and methods for their use in generating customized internal spaces within hydrogels formed by divalent cation cross-linking of hydrogel polymers.

Accordingly in one aspect provided herein is a method for controlled fabrication of internal spaces in a hydrogel (e.g., a biocompatible hydrogel) comprising:

(i) providing a sacrificial template that has a predefined shape and is immobilized within a casting chamber, wherein the sacrificial template comprises a water-soluble thermoplastic-divalent cationic salt composite material; and

(ii) introducing into the casting chamber a volume of solution comprising a hydrogel polymer that is cross-linkable by divalent cations, wherein the volume is sufficient to surround the sacrificial template; whereby, (a) divalent cations diffuse out of the sacrificial template and cross-link the hydrogel polymer in the solution contacting the sacrificial template to form a hydrogel shell conforming to the shape of the sacrificial template; and (b) subsequent to formation of the hydrogel shell, the sacrificial template dissolves to form one or more internal spaces corresponding to the predefined shape.

In some embodiments the water-soluble thermoplastic in the water-soluble thermoplastic-divalent cation composite material is selected from among poly(vinyl alcohol), poly(ethylene-oxide), poly(ethylene glycol), poly(lactic acid), poly(glycolic acid), and combinations thereof. In some embodiments the water-soluble thermoplastic is poly(vinyl alcohol).

In some embodiments wherein the poly(vinyl alcohol)-divalent cationic composite material comprises calcium cations, magnesium cations, or barium cations. In some embodiments the poly(vinyl alcohol)-divalent cationic composite material comprises calcium cations.

In some embodiments the poly(vinyl alcohol)-divalent cationic composite material comprises a calcium salt having a solubility of at least 0.01 g/ml to about 10 g/ml in water at 30° C. In some embodiments the poly(vinyl alcohol)-divalent composite cationic material comprises a calcium salt selected from the group consisting of: calcium acetate, calcium selenate, and calcium formate. In some embodiments the poly(vinyl alcohol)-divalent cationic composite material comprises calcium acetate. In some embodiments, where the calcium salt used is calcium acetate, the poly(vinyl alcohol)-divalent cationic composite material comprises about 5% to about 30% (w/w) calcium acetate. In some embodiments the poly(vinyl alcohol)-divalent cationic composite material comprises about 10% calcium acetate.

In some embodiments hydrogel polymer solution comprises a divalent cation cross-linkable polymer selected from the group consisting of alginate, polysaccharides, xanthan gums, natural gum, agar, agarose, carrageenan, fucoidan, furcellaran, laminaran, hypnea, eucheuma, gum arabic, gum ghatti, gum karaya, gum tragacanth, locust beam gum, arabinogalactan, pectin, amylopectin, and ribo- or deoxyribonucleic acids. In some embodiments the hydrogel polymer solution comprises alginate.

In some embodiments the shape of the one or more internal spaces deviates by no more than about 10% to about 15% from the sacrificial template's predefined shape.

In some embodiments the above controlled fabrication method further includes the step of introducing into the casting chamber a cross-linking solution comprising divalent cations at a concentration sufficient to crosslink the hydrogel polymer, whereby remaining free hydrogel polymer in the volume of solution is cross-linked to form a hydrogel monolith comprising the one or more internal spaces.

In some embodiments the remaining free hydrogel polymer is cross-linked to form the hydrogel monolith at a temperature of about 4° C. to about 45° C. In some embodiments the remaining free hydrogel polymer is cross-linked to form the hydrogel monolith at a temperature of about 37° C.

In some embodiments the hydrogel polymer solution introduced into the casting chamber further comprises a divalent cation at a concentration sufficient to partially cross-link the hydrogel polymer.

In some embodiments the hydrogel polymer solution further comprises a plurality of live cells.

In another aspect described herein is a method for generating a sacrificial template having a predefined shape, comprising injecting a poly(vinyl alcohol)-divalent cation composite material into a mold comprising an internal space corresponding to the predefined shape to obtain a sacrificial template having the predefined shape, wherein the poly(vinyl alcohol)-cation composite material comprises a divalent cationic salt in an amount sufficient to initiate cross-linking of a divalent cation-hydrogel polymer, but insufficient to cause crosslinking of the poly(vinyl alcohol) cation composite material.

In some embodiments the divalent cationic salt to be used for generating the sacrificial template is a calcium salt, a magnesium salt, or a barium salt. In some embodiments the divalent cationic salt to be used is a calcium salt. In some embodiments, where the poly(vinyl alcohol)-cation composite material comprises a calcium salt, the calcium salt is calcium acetate.

In some embodiments the poly(vinyl alcohol)-calcium composite material comprises about 5-20% (w/w) calcium acetate. In some embodiments the poly(vinyl alcohol)-calcium composite material comprises about 10% (w/w) calcium acetate.

In some embodiments the ductility of the sacrificial template is about 1.0 mm/mm to about 2.0 mm/mm strain at break; or the sacrificial template has a Young's Modulus of about 500 MPa to about 900 MPa.

In another aspect described herein is a sacrificial template comprising a water-soluble thermoplastic-divalent cationic salt composite material comprising a divalent cationic salt in an amount sufficient to initiate cross-linking of a divalent cation cross-linkable hydrogel polymer.

In some embodiments the water-soluble thermoplastic-divalent cationic salt composite material is a poly(vinyl alcohol)-divalent cation composite material, and the amount of the divalent cationic salt is insufficient to cause excessive cross-linking of the poly(vinyl alcohol).

In some embodiments the divalent cationic salt is a calcium salt, a magnesium salt, or a barium salt. In some embodiments the sacrificial template comprises a calcium salt. In some embodiments the poly(vinyl alcohol)-divalent cationic salt composite material is a poly(vinyl alcohol)-calcium salt composite material. In some embodiments, where the poly(vinyl alcohol)-divalent cation composite material comprises a calcium salt, the calcium salt is calcium acetate.

In some embodiments the poly(vinyl alcohol)-calcium composite material in the sacrificial template comprises about 5-20% (w/w) calcium acetate. In some embodiments the poly(vinyl alcohol)-calcium composite material comprises about 10% (w/w) calcium acetate.

In some embodiments the ductility of the sacrificial template is about 1.0 mm/mm to about 2.0 mm/mm strain at break; or the sacrificial template has a Young's Modulus of about 500 MPa to about 900 MPa.

In some embodiments the above-mentioned calcium cross-linkable hydrogel polymer comprises a divalent cation cross-linkable polymer selected from the group consisting of alginate, polysaccharides, xanthan gums, natural gum, agar, agarose, carrageenan, fucoidan, furcellaran, laminaran, hypnea, eucheuma, gum arabic, gum ghatti, gum karaya, gum tragacanth, locust beam gum, arabinogalactan, pectin, amylopectin, and ribo- or deoxyribonucleic acids. In some embodiments the calcium cross-linkable hydrogel polymer comprises alginate.

In some embodiments any of the above-mentioned sacrificial templates are obtained by injection molding of the water-soluble thermoplastic-divalent cation composite material.

In a related aspect described herein is a kit comprising any of the above-mentioned sacrificial templates and a divalent cation cross-linkable hydrogel polymer. In some embodiments the divalent cross-linkable hydrogel polymer included in the kit is a calcium cross-linkable hydrogel polymer. In one embodiment the calcium cross-linkable hydrogel polymer is alginate.

In some embodiments the sacrificial template included in the kit comprises one or more modular connectors that permits interconnection with one or more additional sacrificial templates.

In some embodiments the kit comprises multiple sacrificial templates comprising one or more modular connectors that permits their interconnection to form a contiguous modular sacrificial template comprising at least two of the plurality of sacrificial templates comprising one or more modular connectors.

### INCORPORATION BY REFERENCE

All publications, patents, and patent applications mentioned in this specification are herein incorporated by reference to the same extent as if each individual publication, patent, and patent application was specifically and individually indicated to be incorporated by reference.

### BRIEF DESCRIPTION OF THE DRAWINGS

The present invention will be better understood and features, aspects and advantages other than those set forth above will become apparent when consideration is given to the following detailed description thereof. Such detailed description makes reference to the following drawings, wherein:

FIG. 1 shows a schematic illustration of poly(vinyl alcohol) and calcium salt compounding followed by injection molding of a sacrificial poly(vinyl alcohol)-calcium template, which is subsequently encapsulated and dissolved within a hydrogel matrix.

FIG. 2A-D shows representative images of channel laden hydrogels, of various hydrogel materials, casted using sacrificial poly(vinyl alcohol) and poly(vinyl alcohol)-CaCl₂ templates with channel structure highlighted by a colored dye fill. FIG. 2A PEG, FIG. 2B polyacrylamide and FIG. 2C alginate hydrogels cast using poly(vinyl alcohol) lattices. FIG. 2D Alginate hydrogel cast using a 10% (w/w) loaded poly(vinyl alcohol)-CaCl₂ template. FIG. 2E Schematic of cross-sectional hydrogel slices made to measure outer, middle, and interior channel diameters. FIG. 2F-G Representative images of cross-sectioned alginate hydrogels cast using FIG. 2F poly(vinyl alcohol) and FIG. 2G 10% loaded poly(vinyl alcohol)-CaCl₂ templates. FIG. 2H Metrological analysis of the hydrogel channel diameter's deviation (%) from the sacrificial template's geometry. Data combined from duplicate experiments to yield n=8, 16 and 24 for outer, middle and inner channels, respectively. Error bars represent standard deviation; *P<0.05; 3 mm scale bar.

FIG. 3A-C shows graphs illustrating the analysis of the mechanical properties of poly(vinyl alcohol) and exemplary embodiments of various poly(vinyl alcohol)-Ca salt composite materials at 25° C., and in particular a tensile test of injection molded poly(vinyl alcohol) and poly(vinyl alcohol)-Ca: FIG. 3A ductility, FIG. 3B Young's modulus and UTS results, n=10 experimental replicates and error bars represent standard deviation. FIG. 3C Rheological analysis of each specimens' viscosity over a range of shear rates.

FIG. 4A-D Material properties and calcium release analysis of neat poly(vinyl alcohol) versus various poly(vinyl alcohol)-Ca salt composite materials. FIG. 4A Representative thermogravimetric scans, n=3 experimental replicates. FIG. 4B Complexometric calcium titration, n=5 experimental replicates, *P<0.05. Differential scanning calorimeter's FIG. 4C second heating and FIG. 4D first cooling curve traces of neat and extruded (Ext) poly(vinyl alcohol) and poly(vinyl alcohol)-calcium composites.

FIG. 5A-C shows representative images of FIG. 5A 10% (w/w) Ca(C₂H₃O₂)₂ loaded poly(vinyl alcohol) sacrificial fiber templates as micro-injection molded, FIG. 5B dissolved within alginate hydrogels and filled with red dye, and FIG. 5C a CT scan reconstruction of the molded channel. Scale bars 3 mm. FIG. 5D Dimensional analysis was performed on CT scan reconstructions of three separately cast alginate hydrogels at 10 points along each micro-channel. Error bar represent standard deviation.

FIG. 6A-G Depicts an exemplary embodiment of modular 3D sacrificial molding using poly(vinyl alcohol)-calcium acetate templates. FIG. 6A Exploded CAD model of modular, 3D template design. FIG. 6B As molded and assembled 3-D template, and FIG. 6C sacrificially molded channel network within alginate hydrogel. FIG. 6D-G A CT scanned reconstruction of the channel network within alginate monolith showing FIG. 6D top, FIG. 6E isometric, FIG. 6F front, and FIG. 6G right views. Scale bar 3 mm.

FIG. 7A-C shows a CAD drawing of an exemplary 2D, sacrificial poly(vinyl alcohol) lattice templates with FIG. 7A front, FIG. 7B top, and FIG. 7C side views shown. FIG. 7D shows a photograph (top view) of an associated CNC-milled aluminum mold for casting 2D lattice templates.

FIG. 8A-B shows a CAD drawing of exemplary 2D, sacrificial filament templates with FIG. 8A front and FIG. 8B side views shown. FIG. 8C A photograph (top view) of an associated CNC-milled aluminum mold for casting 2D sacrificial filament templates.

FIG. 9A-D shows a CT scanned reconstruction of a 3D channel network cast using poly(vinyl-alcohol)-calcium acetate templates depicted in FIG. 6 but within a polyacrylamide monolith. FIG. 9A top, FIG. 9B isometric, FIG. 9C front, and FIG. 9D right views are shown.

### DETAILED DESCRIPTION

Disclosed herein are poly(vinyl alcohol)-cationic sacrificial templates, methods for making them, and methods for their use in generating customized internal space architectures within biocompatible hydrogels.

“about,” as used herein refers to a value within 3% of a stated figure.

“biocompatible,” as used herein, means not substantially interfering with the viability of cells (e.g., mammalian cells) or tissues.

“hydrogel cross-linking agent,” or “hydrogel cross-linker,” as used herein, refer to an agent (e.g., calcium cations) that initiates cross-linking of hydrogel polymers (e.g., alginate polymers) into a hydrogel. A suitable hydrogel cross-linking agent is selected based upon the specific type of hydrogel polymer to be cross-linked. For example, for divalent cross-linkable hydrogel polymers such as alginate, divalent cations such as calcium, magnesium, or barium may be used. In other cases, depending on the selected hydrogel polymer, the hydrogel cross-linker may be an anionic agent or a free-radical generator/initiator.

“excessive cross-linking,” as used herein, refers to a level of cross-linking within a water-soluble thermoplastic-divalent cation composite material (e.g., poly(vinyl alcohol)-calcium such that its viscosity is higher than about three fold that of the corresponding neat thermoplastic material (e.g., neat poly(vinyl alcohol) at shear rates of 0 to 0.1 (1/s).

“free hydrogel polymer,” or “hydrogel polymer solution,” or “free hydrogel polymer solution,” as used herein, refers to free hydrogel polymer in solution (e.g., alginate) prior to the hydrogel polymer being cross-linked to form a hydrogel.

“hydrogel polymer,” as used herein refers to a polymer that is cross-linkable with other such polymers to form a hydrogel. For example, a hydrogel polymer can be a divalent cation cross-linkable hydrogel polymer such as alginate.

a “hydrogel monolith” refers to a volume of hydrogel obtained by sustained cross-linking of a hydrogel polymer by divalent cations (e.g., calcium) within a casting chamber.

a “hydrogel shell,” as used herein refers to a hydrogel layer formed by cross-linking by initial diffusion of divalent cations from a sacrificial template to the polymer solution immediately contacting the sacrificial template.

a “predefined shape” as used herein refers to a sacrificial template shape that defines a volume which excludes hydrogel polymer cross-linking to define an internal space corresponding to the shape of the sacrificial template within a hydrogel monolith.

a “water-soluble thermoplastic-divalent cation composite material,” as used herein refers to any of a number of composite materials obtained by compounding water-soluble thermoplastics (e.g., poly(vinyl alcohol) with a salt of a divalent cation (e.g., a calcium salt, a magnesium salt, or a barium salt). Optionally, a water-soluble thermoplastic-divalent composite material may include additional components, e.g., ionic chelators such as EGTA that affect the concentration of free cations (e.g., calcium cations) in the composite material or in solution upon dissolution of the composite material in a solvent (e.g., a hydrogel polymer solution). Depending on the type of hydrogel to be used, a water-soluble thermoplastic-divalent composite material may include synthetic free-radical generators/initiators to induce bulk polymerization of, e.g., a methacrylated hydrogel polymer solution upon release.

### I. Compositions

Described herein are sacrificial templates comprising a water-soluble thermoplastic-divalent cationic salt composite material, wherein the amount of the divalent cationic salt in the water-soluble thermoplastic-divalent cation composite material is sufficient to initiate cross-linking of a cation-cross-linkable hydrogel material. Examples of suitable water-soluble thermoplastics include, but are not limited to, poly(vinyl alcohol), poly(ethylene-oxide), poly(ethylene glycol), poly(lactic acid), poly(glycolic acid), and combinations thereof. In some embodiments, where the water-soluble thermoplastic is a susceptible to cross-linking by divalent cations (e.g., poly(vinyl alcohol)), the amount of the divalent cationic salt sufficient to initiate cross-linking of the divalent cation cross-linkable hydrogel polymer is not an amount sufficient to cause excessive crosslinking of the water-soluble thermoplastic. In some embodiments the water-soluble thermoplastic is poly(vinyl alcohol). The divalent cation content of the sacrificial templates described herein is balanced between two criteria. A divalent cation content that is too high results in excessive cross-linking of certain water soluble thermoplastic materials such as poly(vinyl alcohol) and causes production failure during injection molding. On the other hand, if the cation content of the poly(vinyl alcohol)-divalent cation composite material is too low, the resulting sacrificial template will fail to cross-link a cation-cross-linkable hydrogel material (e.g., alginate) at a rate high enough to accurately capture the shape of the sacrificial template as a internal space within the cross-linked hydrogel. In other words, the volume of the surrounding uncross-linked hydrogel material will erode the sacrificial template before the hydrogel has cross-linked thereby resulting in a distorted final internal space shape. Those skilled in the art will appreciate that in some embodiments the concentration of a free divalent cation available for cross-linking can be regulated by including within a water-soluble thermoplastic-divalent cation composite material a chelating compound that chelates free divalent cation thereby reducing the concentration of free divalent cation available to cross-link. This approach provides an alternative to merely changing the total amount of divalent cationic salt included in the thermoplastic-divalent cation composite material to optimize production of a sacrificial template and accuracy of internal space architectures created with a sacrificial template. For example, EGTA is a well known chelator that binds with high specificity and affinity to free calcium cations, and can therefore be used to control the total amount of calcium available for cross-linking.

Accordingly, in some embodiments substantially all of the mass of a sacrificial template is made of the poly(vinyl alcohol)-divalent cationic salt composite material. In other embodiments, the sacrificial templates may include other components, e.g., a dye, a cation chelator (e.g. EDTA or EGTA), a hydrogel cross-linker (e.g. a free radical generator/initiator or anionic molecule or polymer), a culture medium supplement etc.

In some embodiments the percent (w/w) of a cationic salt in the poly(vinyl alcohol)-divalent cation composite material ranges from about 5% to about 30%, e.g., 6%, 7%, 8%, 9%, 10%, 11%, 12%, 15%, 17%, 20%, 23%, 25%, 27%, or another percent (w/w) from about 5% to about 30%. In some embodiments the percent (w/w) of a divalent cationic salt ranges from about 7% to about 15%. In other embodiments the percent (w/w) of a divalent cationic salt in the poly(vinyl alcohol)-divalent cation composite material is about 10%. In some embodiments, the poly(vinyl alcohol)-divalent cation composite material comprises a divalent cationic salt the solubility of which ranges from at least 0.01 g/ml to about 10 g/ml in water at 30° C., e.g., about 0.03 g/ml, 0.05 g/ml, 0.10 g/ml, 0.20 g/ml, 0.3 g/ml, 0.5 g/ml, 0.7 g/ml, 0.8 g/ml or another solubility value ranging from at least 0.01 g/ml to about 1.0 g/ml in water at 30° C. In some embodiments the poly(vinyl alcohol)-divalent cation composite material is a poly(vinyl alcohol)-calcium salt composite material, a poly(vinyl alcohol)-magnesium salt composite material, or a poly(vinyl alcohol)-barium salt material. In some embodiments the poly(vinyl alcohol)-divalent cation composite material is a poly(vinyl alcohol)-calcium salt composite material. In some embodiments the poly(vinyl alcohol)-calcium salt composite material includes a calcium acetate, calcium selenate, or calcium formate salt. In some embodiments the calcium salt in the poly(vinyl alcohol)-calcium salt composite material is calcium acetate. In some embodiments the poly(vinyl alcohol)-calcium acetate composite material comprises about 10% (w/w) calcium acetate.

One advantage of the sacrificial templates comprising a water-soluble thermoplastic-divalent cation composite material (e.g., poly(vinyl alcohol)-calcium) as described herein is their superior mechanical resilience relative to sacrificial templates generated using certain other water-soluble materials such as carbohydrate glass, which is quite brittle and prone to damage during transport or use. In some embodiments the sacrificial templates described herein have a ductility of about 1.0 mm/mm to about 2.0 mm/mm strain at break, e.g., 1.2, 1.4, 1.6, 1.7, 1.8, or another ductility value from about 1.0 mm/mm to about 2.0 mm/mm strain at break. In some embodiments the sacrificial template has a Young's modulus of about 500 MPa to about 900 MPa, e.g., about 520 MPa, 540 MPa, 600 MPa, 650 MPA, 700 MPA, 750 MPA, 800 MPA, 830 MPA, 870 MPa, or another Young's modulus value from about 500 MPA to about 900 MPA.

The sacrificial template can be of any shape. In some embodiments the sacrificial template comprises one or more hollow structures. In other embodiments the sacrificial template does not comprise hollow structures.

In some embodiments a sacrificial template described herein is provided in a kit comprising the sacrificial template and a hydrogel polymer (e.g., alginate) that is cross-linkable by divalent cations (e.g., calcium). In some embodiments, the uncross-linked hydrogel material is cross-linkable by calcium cations. In some embodiments the hydrogel polymer comprises one selected from among alginate, polysaccharides, xanthan gums, natural gum, agar, agarose, carrageenan, fucoidan, furcellaran, laminaran, hypnea, eucheuma, gum arabic, gum ghatti, gum karaya, gum tragacanth, locust beam gum, arabinogalactan, pectin, amylopectin, and ribo- or deoxyribonucleic acids, and combinations thereof. In some embodiments the hydrogel polymer included in a kit is alginate.

In further embodiments a kit comprising a sacrificial template and a hydrogel polymer includes a plurality of sacrificial templates comprising one or more modular connectors that permit their interconnection to form a contiguous modular sacrificial template comprising at least two of the plurality of sacrificial templates comprising one or more modular connectors.

II. Methods for Generating Sacrificial Templates from Water-Soluble-Divalent Cation Composite Materials

Also described herein is a method for generating a sacrificial template having a predefined shape, comprising injecting a water-soluble thermoplastic-divalent cation composite material into a mold comprising an internal space corresponding to the predefined shape to obtain a sacrificial template having the predefined shape, wherein the water-soluble thermoplastic-divalent cation composite material comprises a divalent cationic salt in an amount sufficient to initiate cross-linking of a divalent cation-cross-linkable hydrogel polymer. Examples of suitable water-soluble thermoplastics include, but are not limited to, poly(vinyl alcohol), poly(ethylene-oxide), poly(ethylene glycol), poly(lactic acid), poly(glycolic acid), and combinations thereof. In some embodiments the water-soluble thermoplastic is poly(vinyl alcohol). Also described herein is a method for generating a sacrificial template having a predefined shape, comprising injecting a poly(vinyl alcohol)-cation composite material into a mold comprising an internal space corresponding to the predefined shape to obtain a sacrificial template having the predefined shape, wherein the poly(vinyl alcohol)-divalent cation composite material comprises a divalent cationic salt in an amount sufficient to initiate cross-linking of a cation-cross-linkable hydrogel polymer, but insufficient to cause excessive crosslinking of the poly(vinyl alcohol)-divalent cation composite material. Advantageously, the methods for generating a sacrificial template, as described herein, utilize injection molding (e.g., microinjection molding), a more rapid method than commonly used fabrication techniques such as 3D printing, which rely on layer by layer deposition of materials. Thus, the use of injection molding is particularly suitable to mass production of the poly(vinyl alcohol)-divalent cationic sacrificial templates described herein.

As described above, it is important that that the divalent cation content of a poly(vinyl alcohol)-divalent cation composite material used herein not be sufficient to induce excessive crosslinking of the poly(vinyl alcohol)-divalent cation composite material, especially during the mold injection process, as excessive crosslinking of the poly(vinyl alcohol)-divalent cation composite material greatly increases its viscosity, which may impede injection of the material into a sacrificial template mold.

Prior to injection molding, a water-soluble thermoplastic-divalent cation composite material, such as a poly(vinyl alcohol)-calcium salt composite, is generated by compounding the water-soluble thermoplastic with a divalent cation salt. In some embodiments the composite material is compounded by thermal extrusion and static mixing.

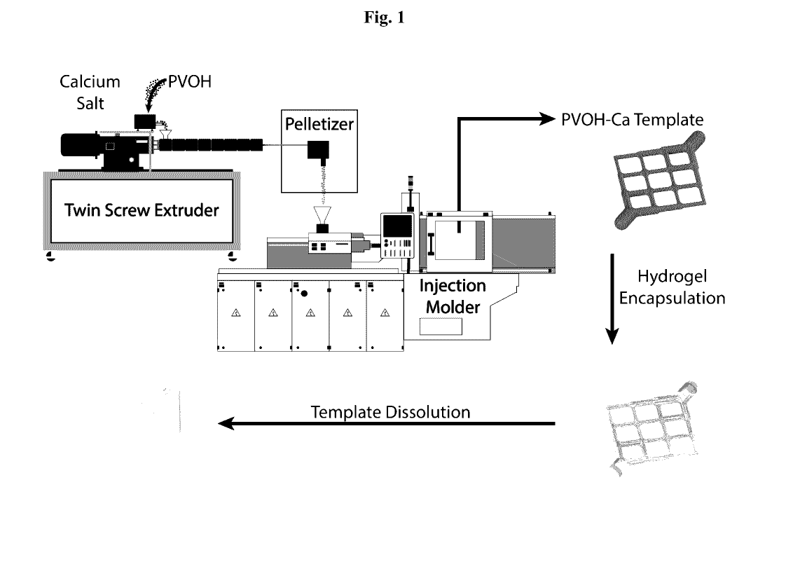

In an exemplary embodiment a divalent cationic salt is ground via mortar and pestle and sieved until less than 300 μm in dimension. Afterwards, the sieved salt is hand mixed with poly(vinyl alcohol) (available commercially, e.g., as Monopol C100, Monosol Inc. or Mowiflex 232, Kuraray Inc.) and conditioned under vacuum at 70° C. for 4 hours. The mixture is then fed through a volumetric feeder (Tuf-Flex 100, Schenck Accurate) into a co-rotating twin screw compounding extruder (18 mm Leistritz) equipped with a screw designed with only feedforward elements and a 6 mm strand die, as schematically illustrated in FIG. 1. Compounding parameters are listed in Table 1.

TABLE 1
Exemplary Twin Screw Extruder Processing Parameters

| | |
|---|---|
| Zone 1 (° C.) | 160 ± 10 |
| Zone 2 (° C.) | 165 ± 10 |
| Zone 3 (° C.) | 170 ± 10 |
| Zone 4 (° C.) | 173 ± 10 |
| Zone 5 (° C.) | 175 ± 10 |
| Zone 6 (° C.) | 177 ± 10 |
| Zone 7 (° C.) | 178 ± 10 |
| Zone 8 (° C.) | 180 ± 10 |
| Load (%) | <40 |
| Screw Speed (rpm) | 50-70 |

Post compounding, the extrudate is run through a pelletizer to prepare the composite for injection molding.

In some exemplary embodiments an injection mold, suitable for generation of sacrificial templates as described herein can be designed with 3D modeling software (Solidworks 2014, Dassault Systemes) and fabricated from 6061 aluminum with a computer numerical control (CNC) vertical machining center (MiniMill 2, Haas) programmed with computer aided design/manufacturing (CAD/CAM) software (MasterCAM X7, CNC Software, Inc.). In an exemplary embodiment injection molding is performed on a 38 ton Arburg Allrounder 270A machine with an 18 mm injection unit with processing parameters found in Table 2.

TABLE 2
Exemplary Injection Molding Process Parameters

| | |
|---|---|
| Mold temperature (° C.) | 46 ± 10 |
| Barrel temperature (° C.) | 190-200 |
| Injection pressure (bar) | 1250-1400 |
| Holding pressure (bar) | 1000 ± 200 |
| Injection speed (cm³/s) | 55 ± 10 |
| Holding time (s) | 2 ± 2 |
| Cooling time (s) | 20 ± 5 |

In some embodiments the divalent cationic salt used in the method is a calcium salt, a magnesium salt, or a barium salt. In some embodiments the percent (w/w) of a cationic salt in the poly(vinyl alcohol)-divalent cation composite material ranges from about 5% to about 30%, e.g., 6%, 7%, 8%, 9%, 10%, 11%, 12%, 15%, 17%, 20%, 23%, 25%, 27%, or another percent (w/w) from about 5% to about 30%. In some embodiments the percent (w/w) of a cationic salt ranges from about 7% to about 15%. In other embodiments the percent (w/w) of a divalent cationic salt in the poly(vinyl alcohol)-divalent cation composite material is about 10%. In some embodiments, the poly(vinyl alcohol)-divalent cation composite material comprises a divalent cationic salt the solubility of which ranges from at least 0.01 g/ml to about 10 g/ml in water at 30° C., e.g., about 0.03 g/ml, 0.05 g/ml, 0.10 g/ml, 0.20 g/ml, 0.3 g/ml, 0.5 g/ml, 0.7 g/ml, 0.8 g/ml or another solubility value ranging from at least 0.01 g/ml to about 1.0 g/ml in water at 30° C. In some embodiments the poly(vinyl alcohol)-divalent cation composite material is a poly(vinyl alcohol)-calcium salt composite material, a poly(vinyl alcohol)-magnesium salt composite material, or a poly(vinyl alcohol)-barium salt material. In some embodiments the poly(vinyl alcohol)-divalent cation composite material is a poly(vinyl alcohol)-calcium salt composite material. In some embodiments the poly(vinyl alcohol)-calcium salt composite material includes a calcium acetate salt, calcium selenate salt, or calcium formate salt. In some embodiments the calcium salt in the poly(vinyl alcohol)-calcium salt composite material is calcium acetate. In some embodiments the poly(vinyl alcohol)-calcium acetate composite material comprises about 10% (w/w) calcium acetate.

The methods for generating a sacrificial template described herein yield sacrificial templates having favorable mechanical properties relative to sacrificial templates generated by other means or with other materials. Accordingly, in some embodiments the sacrificial templates generated by the methods described herein have a ductility of about 1.0 mm/mm to about 2.0 mm/mm strain at break, e.g., 1.2, 1.4, 1.6, 1.7, 1.8, or another ductility value from about 1.0 mm/mm to about 2.0 mm/mm strain at break. In some embodiments the sacrificial template has a Young's modulus of about 500 MPa to about 900 MPa, e.g., about 520 MPa, 540 MPa, 600 MPa, 650 MPA, 700 MPA, 750 MPA, 800 MPA, 830 MPA, 870 MPa, or another Young's modulus value from about 500 MPA to about 900 MPA.

III. Methods for Custom Generation of Internal Space Architectures within Hydrogels

Also described herein are methods for controlled fabrication of internal spaces in a hydrogel (e.g., a biocompatible hydrogel), where the method includes at least the steps of:

(i) providing a sacrificial template that has a predefined shape and is immobilized within a casting chamber, wherein the sacrificial template comprises a water-soluble thermoplastic-divalent cation composite material; and

(ii) introducing into the casting chamber a volume of solution comprising a hydrogel polymer that is cross-linkable by divalent cations, wherein the volume is sufficient to surround the sacrificial template; whereby, divalent cations diffuse out of the sacrificial template and cross-link the hydrogel polymer in the solution contacting the sacrificial template to form a hydrogel shell conforming to the shape of the sacrificial template; and (b) subsequent to formation of the hydrogel shell, the sacrificial template dissolves to form one or more internal spaces corresponding to the predefined shape. The controlled fabrication methods described herein allow precise casting of microscale features such as microchannel networks within hydrogels. For example, features of about 500 μM in diameter can be cast with as little as about ±7% deviation in a divalent cation cross-linked hydrogel such as an alginate hydrogel.

In some of the embodiments the water-soluble thermoplastic material in the just-mentioned water-soluble thermoplastic-divalent cation composite material is selected from among poly(vinyl alcohol), poly(ethylene-oxide), poly(ethylene glycol), poly(lactic acid), poly(glycolic acid), and combinations thereof. In some embodiments the water-soluble thermoplastic used is poly(vinyl alcohol).

In some embodiments of the above method substantially all of the mass of a sacrificial template to be used is made of the water-soluble thermoplastic-divalent cation composite material. In other embodiments, a sacrificial template includes other components, e.g., a dye, a cation chelator(such as EDTA or EGTA), a cross-linker (e.g. a free radical generator/initiator or anionic molecule or polymer), a culture medium supplement etc. In some embodiments the divalent cationic salt used in the sacrificial template of the above method is a calcium salt, a magnesium salt, or a barium salt. In some embodiments the percent (w/w) of a divalent cationic salt supplying divalent cations in the water-soluble thermoplastic-divalent cation composite material ranges from about 5% to about 30%, e.g., 6%, 7%, 8%, 9%, 10%, 11%, 12%, 15%, 17%, 20%, 23%, 25%, 27%, or another percent (w/w) from about 5% to about 30%. In some embodiments the percent (w/w) of a divalent cationic salt ranges from about 7% to about 15%. In other embodiments the percent (w/w) of a divalent cationic salt in the water-soluble thermoplastic-divalent cation composite material is about 10%.

In some embodiments the sacrificial template comprises a poly(vinyl alcohol)-divalent cation composite material. In some embodiments the poly(vinyl alcohol)-divalent cation composite material is a poly(vinyl alcohol)-calcium composite material. In some embodiments, the poly(vinyl alcohol)-divalent cation composite material comprises a divalent cationic salt the solubility of which ranges from at least 0.01 g/ml to about 10 g/ml in water at 30° C., e.g., about 0.03 g/ml, 0.05 g/ml, 0.10 g/ml, 0.20 g/ml, 0.3 g/ml, 0.5 g/ml, 0.7 g/ml, 0.8 g/ml or another solubility value ranging from at least 0.01 g/ml to about 1.0 g/ml in water at 30° C. In some embodiments the poly(vinyl alcohol)-divalent cation composite material is a poly(vinyl alcohol)-calcium salt composite material, a poly(vinyl alcohol)-magnesium salt composite material, or a poly(vinyl alcohol)-barium salt material. In some embodiments the poly(vinyl alcohol)-divalent cation composite material is a poly(vinyl alcohol)-calcium salt composite material. In some embodiments the poly(vinyl alcohol)-calcium salt composite material contains calcium acetate, calcium selenate, calcium formate, or a combination thereof. In some embodiments the percent (w/w) of a calcium salt in the poly(vinyl alcohol)-divalent cationic salt composite material ranges from about 5% to about 30%, e.g., 6%, 7%, 8%, 9%, 10%, 11%, 12%, 15%, 17%, 20%, 23%, 25%, 27%, or another percent (w/w) from about 5% to about 30%. In some embodiments the calcium salt in the poly(vinyl alcohol)-calcium salt composite material is calcium acetate. In some embodiments of the method the poly(vinyl alcohol)-calcium acetate composite material used in the sacrificial template comprises about 7% to about 15% (w/w) calcium acetate. In some embodiments the poly(vinyl alcohol)-calcium acetate composite material comprises about 10% (w/w) calcium acetate.

In some embodiments the hydrogel polymer solution has a hydrogel polymer concentration (w/v) of about 0.5% to about 5%, e.g., about 0.7%, 0.8%, 1%, 1.2%, 1.5%, 1.7%, 2.0%, 2.5%, 3.0%, 3.5%, 4.2%, 4.5% or another concentration (w/v) of hydrogel polymer ranging from about 0.5% to about 5%. In some embodiments the hydrogel polymer solution has a concentration of about 2% (w/v) hydrogel polymer. Optionally, the hydrogel polymer solution may include a divalent cation at a concentration suitable for partially cross-linking the hydrogel polymer in the solution such that the viscosity of the solution is higher than that of a polymer solution in the absence of the cross-linking ion (e.g., a hydrogel polymer slurry), but not at a concentration sufficient to cross-link the hydrogel polymer solution into a hydrogel monolith. The advantage of using of a hydrogel polymer slurry rather than a less viscous hydrogel polymer solution is that the former can facilitate faster casting of a hydrogel monolith, though use of a hydrogel polymer slurry is not essential. In some embodiments, where the hydrogel polymer solution to be used includes a divalent cationic salt to form a hydrogel slurry prior to casting, the concentration % (w/v) of the divalent cationic salt (e.g., calcium sulfate) is about 0.1% to about 1%, e.g., 0.2%, 0.3%, 0.4%, 0.5%, 0.6%, 0.7%, 0.8%, or another concentration (w/v) of the divalent cationic salt ranging from about 0.1% to about 1%. In some embodiments the hydrogel polymer solution comprises about 0.5% calcium sulfate.

In some embodiments the hydrogel polymer used in the controlled fabrication method described herein comprises one selected from among alginate, polysaccharides, xanthan gums, natural gum, agar, agarose, carrageenan, fucoidan, furcellaran, laminaran, hypnea, eucheuma, gum arabic, gum ghatti, gum karaya, gum tragacanth, locust beam gum, arabinogalactan, pectin, amylopectin, and ribo- or deoxyribonucleic acids, any of which cross-linked by divalent cations (e.g., calcium ions) form a hydrogel. In some embodiments the uncross-linked hydrogel material is alginate. Alginate can be ionically cross-linked with divalent cations, in water, at room temperature, to form a hydrogel matrix. Due to these mild conditions, alginate has been the most commonly used polymer for cell encapsulation, as described, for example, in U.S. Pat. No. 4,352,883 to Lim. Suitable solvents for hydrogel polymers include, but are not limited to, water, aqueous buffers, cell culture media, and the like. Preferably, hydrogel polymers are dissolved in a sterile solvent and under sterile conditions to avoid microbial contamination, which is especially relevant if viable cells are to be included in a hydrogel polymer solution or hydrogel as described herein.

In some embodiments the resulting shape of the one or more internal spaces fabricated within a hydrogel deviate by no more than about between about 5% to about 20% from the predefined shape, e.g., no more than about 6%, 8%, 10%, 12%, 14%, 15%, 17%, or another percent from about 5% to about 20% deviation from the predefined shape. In some embodiments the one or more internal spaces fabricated within a hydrogel deviate by no more than about 10% to about 15% from the predefined shape.

Casting chambers of varying shapes and sizes may be used to provide external shape to the hydrogel monolith. In some embodiments the casting chamber is a shape selected from among, cuboidal, parallelepipedal, prismoidal, spherical, pyramidal, conical, or any sections thereof. In other embodiments the casting chamber is of a custom designed shape, e.g., to conform to particular tissue or organ shape or a custom space within an or organ or tissue. In some embodiments the volume of the casting chamber ranges from about 0.5 cm³ to about 500 cm³, e.g., about 1 cm³, 2 cm³, 5 cm³, 8 cm³, 10 cm³, 20 cm³, 25 cm³, 30 cm³, 50 cm³, 100 cm³, 150 cm³, 200 cm³, 300 cm³, 400 cm³, 450 cm³, or another volume from about 0.5 cm³ to about 500 cm³. Suitable materials for a casting chamber include, but are not limited to, stainless steel, inert metals or alloys, polypropylene, polystyrene, teflon, and the like. Optionally the casting chamber may include features to facilitate suspension of a sacrificial template for hydrogel casting, e.g. inlet and outlet ports sized for a conformal fit with the ends of the sacrificial template.

In some embodiments the method further includes a step of introducing into the casting chamber a cross-linking solution comprising divalent cations (e.g., calcium cations) at a concentration sufficient to crosslink the hydrogel polymer whereby the remaining free (i.e., not cross-linked) hydrogel polymer in the bulk volume of solution in the casting chamber is cross-linked to form a hydrogel monolith surrounding the one or more internal spaces.

The cross-linking solution may contain the same type of divalent cation contained in the sacrificial template, a different divalent cation, or a combination thereof. In some embodiments the cross-linking solution comprises calcium cations, magnesium cations, or barium cations. In some embodiments the cross-linking solution comprises calcium cations. In some embodiments the cross-linking solution has a divalent cationic salt concentration (w/v) ranging from about 0.5% to about 10%, e.g., about 0.6%, 0.8%, 1.2%, 1.5%, 2.0%, 2.5%, 3.0%, 4%, 5%, 7%, 8%, 9%, or another divalent cationic salt concentration ranging from about 0.5% to about 10%.

In some embodiments, following addition of the cross-linking solution, the remaining free hydrogel polymer is cross-linked to form the hydrogel monolith at a temperature of about 4° C. to about 45° C., e.g., about 4° C., 6° C., 8° C., 10° C., 14° C., 25° C., 27° C., 30° C., 34° C., 37° C., 38° C., 40° C., 42° C., or another temperature from about 4° C. to 45° C. In some embodiments the temperature used is about 25° C. In other embodiments, particularly if living cells are to be cultured within the resulting hydrogel monolith, the temperature to be used is about 37° C.

In some embodiments the solution comprising the hydrogel polymer also includes a plurality of lives cells. Such cells are typically eukaryotic cells. In some embodiments the cells to be included are mammalian cells including, but not limited to, undifferentiated cell types (e.g., induced pluripotent stem cells, embryonic stem cells, and mesenchymal stem cells), as well as differentiated cell types. In some embodiments differentiated cell types to be included in the hydrogel polymer solution for hydrogel casting include neurons, astrocytes, oligodendrocytes, microglia, hepatocytes, cardiomyocytes, muscle cells, kidney cells, endothelial cells, T-cells, lymphocytes, macrophages, or any combination thereof.

The skilled artisan will appreciate that where cells are to be cultured in a hydrogel monolith/scaffold fabricated as described herein, it is necessary that working solutions be kept sterile throughout hydrogel casting (e.g., cross-linking) prior to culture of embedded cells so as to prevent contamination of the cell cultures.

The following specific examples are to be construed as merely illustrative, and not limitative of the remainder of the disclosure in any way whatsoever. Without further elaboration, it is believed that one skilled in the art can, based on the description herein, utilize the present invention to its fullest extent. All publications cited herein are hereby incorporated by reference in their entirety.

### Examples

### Example 1: Materials and Methods for Fabrication of PVC-Calcium Templates

Hydrogel biomaterials are used throughout the field of tissue engineering as versatile scaffolds to support 3D cell growth and shape the morphology of tissue constructs (1-6). In vivo, normal tissue development and physiology relies upon proper cytoarchitectural organization at multiple length scales. Hence, several methods have been developed for engineering the macro-to-microscale architecture of hydrogel scaffolds including layer-by-layer 3D printing technologies, such as fused deposition modeling (FDM), and stereolithography (SLA) (7-10). Recently, these methods have been used to fabricate sacrificial templates that enable unprecedented, rapid casting of intricate architectures within hydrogel monoliths (11, 12). For example, Miller et al. used a FDM printer to create interconnected 3D lattices composed of water-soluble carbohydrate glass filaments (13). Subsequently, the lattices could be encapsulated within hydrogels, and upon dissolution, leave behind channel networks suitable for generating microvasculature within prospective 3D tissues. Alternatively, sacrificial poly(vinyl alcohol) (poly(vinyl alcohol)), alginate, gelatin and PEG templates casted within SLA fabricated molds have also been developed to engineer microscale hydrogel architecture(14-17).

While these approaches enable rapid casting of complex hydrogel architectures, the fabrication techniques and sacrificial template materials impose several limitations. First, the FDM and SLA/solvent casting fabrication techniques are not scalable for mass production due to extended manufacturing cycle times per sacrificial template. Second, the carbohydrate glass template's reported mechanical properties indicate its brittle and inelastic nature, suggesting limited durability during normal handling, whereas those of the poly(vinyl alcohol) and alginate templates were not directly determined. Third, current template materials have only been proven to effectively cast complex geometries within bulk curing hydrogels such as PEG, fibrin and methacrylated gelatin. Poorer dimensional accuracy was observed when Miller et al. used carbohydrate glass lattices to cast channels within diffusion limited, ionically crosslinked alginate hydrogels, a widely used tissue engineering scaffold and clinically approved biomaterial (13, 18-20). Alginate in particular has several advantageous biomaterial properties for tissue engineering applications: facile and gentle cell encapsulation, ease of chemical modification via densely presented carboxylic acid groups on its polymer backbone, and enzymatic degradation mechanisms that are orthogonal to the mammalian genome. Thus, there remains a need to develop a scalable mass production process for generating durable sacrificial templates capable of accurately casting architectural features within both bulk curing and diffusion limited, ionically crosslinked hydrogels.

To address these limitations, we have developed micro-injection molded poly(vinyl alcohol)-calcium salt composites (poly(vinyl alcohol)-Ca) as enhanced sacrificial templates for engineering hydrogel architecture (FIG. 1). Injection molding processes are unmatched in manufacturing scalability due to their automated, parallel production of finished polymer components within seconds(22). Interestingly, calcium salt solubility was discovered to be a critical parameter in optimizing the poly(vinyl alcohol)-Ca's micro-injection molding processability and the resulting templates' handling durability and casting precision. Calcium acetate (Ca(C₂H₃O₂)₂) was determined to induce optimal poly(vinyl alcohol)-Ca composite mechanical properties, and its release upon template dissolution accelerated the curing rate of alginate hydrogels at the template/hydrogel interface. poly(vinyl alcohol)-(Ca(C₂H₃O₂)₂) templates were observed to cast 500 μm diameter features within alginate monoliths with only a 6.4±7.2% average error, and equivalent performance was observed within bulk curing polyacrylamide hydrogels. Also, to demonstrate process scalability and versatility, modular poly(vinyl alcohol)-(Ca(C₂H₃O₂)₂) templates were injection molded, assembled into multicomponent Lego®-like structures, and used to generate 3D channel networks within alginate and polyacrylamide hydrogels. Overall, these results demonstrate the mass-production, utility, and versatility of poly(vinyl alcohol)-calcium acetate templates for sacrificially molding custom architectures within hydrogels used for tissue engineering applications.

### 1. Materials and Methods

1.1 Materials

Pronova SLG1000 sodium alginate was purchased from Pronova Biopolymer. Calcium salts (CaCO₃, Ca(C₂H₃O₂)₂, CaI₂ and CaCl₂), Eriochrome Black T, Ammonium Chloride, and Magnesium Chloride were purchased from Sigma Aldrich. Calcium Sulfate and Ammonia were purchased from Acros Organics. Polyacrylamide, TEMED, and Ammonium Persulfate were purchased from Biorad. PEG hydrogel materials were kindly provided by Prof. William Murphy. Polypropylene 6061 aluminum and 316 stainless steel raw material was purchased from McMaster-Carr Supply Company.

1.2 Extrusion Compounding

Calcium salts were ground via mortar and pestle and sieved until less than 300 μm in dimension. Then, the salts were hand mixed with poly(vinyl alcohol) (Monopol C100, Monosol Inc.) and conditioned under vacuum at 70° C. for 4 hours. The mixture was fed through a volumetric feeder (Tuf-Flex 100, Schenck Accurate) into a co-rotating twin screw compounding extruder (18 mm Leistritz) equipped with a screw designed with only feedforward elements and a 6 mm strand die (FIG. 1). Post compounding, the extrudate was run through a pelletizer to prepare the composite for injection molding. Compounding parameters are listed in Supplemental Table 1.

1.3 Mechanical and Thermal Analysis

Tensile properties were measured using an Instron 5967 Universal Tensile Testing Machine (30 kN) with injection molded ASTM D638 Type V tensile bars elongated at 50 mm/min (31). Thermogravimetric Analysis (TGA) was performed on poly(vinyl alcohol)-Ca composite samples ranging from 20-60 mg with platinum pans (DSC Consumables Inc.) ramped to 600° C. at 10° C./min (Q50 TGA, TA Instruments). Differential scanning calorimetry (DSC) was performed using hermetic aluminum pans (DSC Consumables Inc.) between 0 and 210° C. at a ramp rate of 10° C./min (Auto Q20, TA Instruments). Viscosity characterization was performed with a parallel plate rheometer (AR2000, TA Instruments) using injection molded discs (24.5 mm diameter, 2 mm thick).

1.4 Injection Molding

Custom injection molds were designed with 3D modeling software (Solidworks 2014, Dassault Systemes) and fabricated from 6061 aluminum with a computer numerical control (CNC) vertical machining center (MiniMill 2, Haas) programmed with computer aided design/manufacturing (CAD/CAM) software (MasterCAM X7, CNC Software, Inc.). All injection molding was performed on a 38 ton Arburg Allrounder 270A machine with an 18 mm injection unit with processing parameters found in Supplemental Table 1.

1.5 Complexometric Calcium Titration

A complexometric titration was performed to quantify the amount of Ca ions released from poly(vinyl alcohol)-Ca templates upon dissolution. First, 1.4 g of each composite was completely dissolved in 50 ml of deionized water. Second, 100 mL of 50 mM EDTA was added to the solution to chelate all free Ca atoms. Third, Eriochrome Black T (EBT), a weaker chelating agent used as an indicator, was added to the solution causing a blue color that changes to pink upon its complexing with cations. Then, MgCl₂ was titrated in the solution to react with the excess EDTA to completion, which was indicated by the solution's color change due to Mg's reaction with EBT. In this manner, we could calculate the amount of Ca released upon poly(vinyl alcohol)-Ca template dissolution from the amount of MgCl₂ added. For each template composition, the titration results were compared with the total Ca salt content detected by TGA.

1.6 Hydrogel Fabrication

1.6.1 Polyacrylamide

Polyacrylamide hydrogels were fabricated within custom polypropylene devices that suspended poly(vinyl alcohol) templates between inlet and outlet needles. Five milliliters of 12% Acrylamide/Bisacrylamide was prepared, and 100 uL of Ammonium Persulfate and 4 uL of TEMED were added to initiate the bulk cross-linking reaction. After 5 min, the polyacrylamide solution was added to the device to completely cover the poly(vinyl alcohol) templates. The solution was allowed to cross-link for one hour to form a hydrogel around the templates. The device was transferred to a water bath at 45° C. and incubated overnight for poly(vinyl alcohol) dissolution.

1.6.2 Polyethylene Glycol (PEG)

PEG hydrogels were fabricated within custom polypropylene devices that suspended poly(vinyl alcohol) templates between inlet and outlet needles. Five milliliters of X % PEG methacrylate was prepared. Immediately, the solution was added to the device to completely cover the poly(vinyl alcohol) templates. The device was placed under a UV lamp at Xum wavelength for 20 seconds to allow cross-linking. The device was transferred to a water bath at 45° C. and incubated overnight for poly(vinyl alcohol) dissolution.

1.6.3 Alginate

Alginate hydrogels were fabricated within custom polypropylene and stainless steel (SS) devices that suspended poly(vinyl alcohol) templates between inlet and outlet ports. To form the alginate hydrogels, a 5 mL solution of 2% sodium alginate in sterile, deionized water was prepared by overnight mixing at 4° C. A CaSO₄-alginate slurry was prepared by adding 300 μL of 7.5% CaSO₄ in deionized water to the 5 mL of 2% sodium alginate solution. The slurry was poured into the device to cover the suspended poly(vinyl alcohol) template, and allowed to pre-gel for 10 min. Then the device was submerged in a 2% (w/v) solution of CaCl₂ in deionized water to finalize the hydrogel gelation. For cell culture experiments, the encapsulated poly(vinyl alcohol) templates were dissolved in a 37° C. water bath for 1 day. Otherwise, they were dissolved overnight at 45° C.

1.7 Imaging and Reconstruction

Hydrogels were imaged using standard photography or a microCATII microCT (Siemens AG.) at the University of Wisconsin Carbone Cancer Center's small animal imaging facility. DICOM image stacks were then reconstructed and converted into STL graphic bodies using Mimics software (Materialize NV.). Image analysis was performed using a combination of Magics (Materialize NV.), MeshLabs and Solidworks (Dassault Systemes).

### Results and Discussion

### 3.1 Injection Molded, Sacrificial Poly(Vinyl Alcohol) and Poly(Vinyl Alcohol)-CaCl₂ Templates

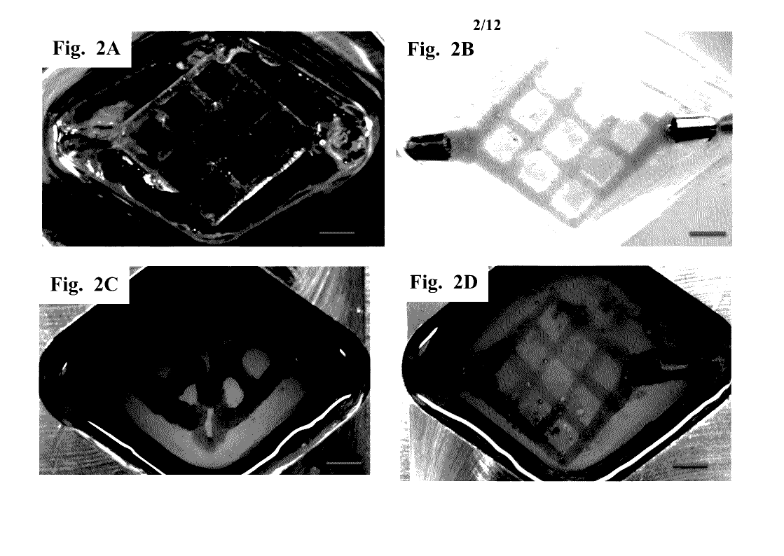

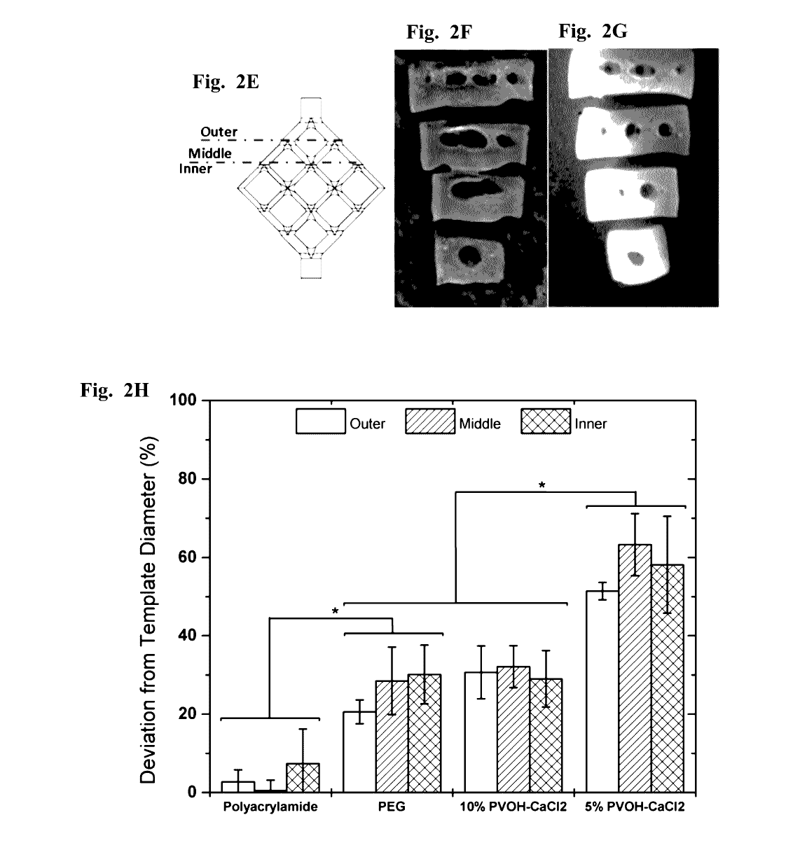

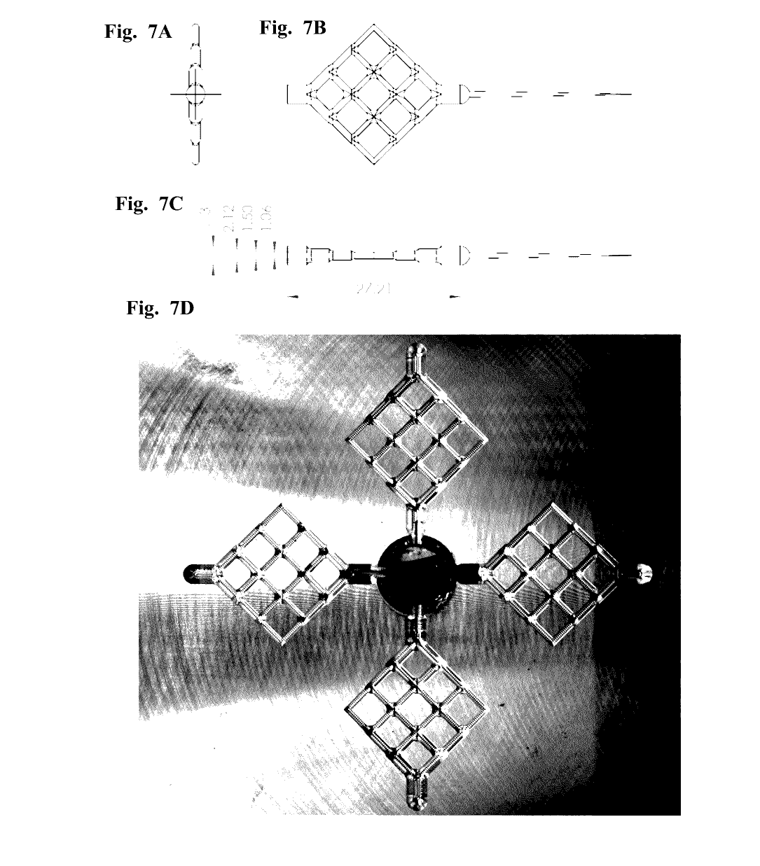

To alleviate handling durability issues inherent to carbohydrate glass sacrificial templates, we opted to produce our templates from water-soluble, biocompatible poly(vinyl alcohol) similar to Tocchio et al (14). Micro-injection molding was chosen as the preferred fabrication technique to mitigate manufacturing mass-production scalability concerns with rapid prototyping based and SLA/solvent casting approaches. Aluminum molds were CNC-milled to produce sacrificial poly(vinyl alcohol) templates with a lattice geometry consisting of cylinders transitioning from 3 to 1 mm in diameter (FIGS. 1, 2E, and 7). poly(vinyl alcohol) lattice templates were injection molded, encapsulated within polyacrylamide, PEG, and alginate hydrogels, and dissolved in a water bath to generate monoliths with replicate internal architecture. Post template dissolution, bulk curing polyacrylamide and PEG hydrogels displayed discernable and precisely casted internal channels (FIG. 2A-B). The PEG hydrogel's channels displayed a higher deviation from the poly(vinyl alcohol) template's dimensions presumably due to the hydrogel's characteristic swelling (FIG. 2H). However, a series of disconnected and non-uniform voids were generated in alginate hydrogels, suggesting that the poly(vinyl alcohol) template dissolved prior to the inward diffusion of Ca²⁺ ions (FIGS. 2C and E-F).

To enable effective sacrificial molding of alginate hydrogels, we hypothesized that compounding calcium salts into the poly(vinyl alcohol) template would accelerate the crosslinking rate at the template/hydrogel interface and result in greater geometric control of the cast internal architecture. poly(vinyl alcohol) was compounded with 5% and 10% (w/w) CaCl₂ using twin screw extrusion, and the poly(vinyl alcohol)-CaCl₂ composite was injection molded into a lattice geometry (FIG. 2E). Upon casting with poly(vinyl alcohol)-CaCl₂ templates, continuous channel networks were observed within alginate hydrogels indicating release of the compounded calcium ameliorated casting efficacy (FIG. 2D-G). Metrological analysis of the sectioned hydrogels (FIG. 2E-G) revealed that compounding 10% vs. 5% (w/w) CaCl₂ into the poly(vinyl alcohol) lattice yielded significantly better casting of channel network geometries (FIG. 2H). However, the casted architecture still displayed a ˜30% dimensional deviation from the poly(vinyl alcohol)-CaCl₂ template. Thus, the addition of CaCl₂ to poly(vinyl alcohol) template enables casting of architecture within both bulk curing and ionically crosslinked hydrogel monoliths, but casting precision within alginate hydrogels could still be improved.

### 3.2 Optimizing Mechanical Properties of Poly(Vinyl Alcohol)-Ca Templates

Although the poly(vinyl alcohol)-CaCl₂ casting results were promising, the addition of CaCl₂ into the poly(vinyl alcohol) substrate also yielded undesirable fabrication side effects. Notably, the composite was much more difficult to process via extrusion and injection molding than neat poly(vinyl alcohol); thermal degradation of the polymer was apparent both visually and aromatically. According to a prior cement study, dissolved Ca²⁺ ions can produce strong crosslinking-like interactions between adjacent hydroxyl groups on poly(vinyl alcohol) backbone chains (23). Since CaCl₂ solubility is high and increases with temperature (Supplemental Table 2), we suspected that the thermal processing of extrusion and injection molding amplified Ca²⁺ crosslinking within the polymer composite. This would make microscale poly(vinyl alcohol)-CaCl₂ templates difficult to injection mold, and it would decrease the Ca²⁺ ions released upon template dissolution and thereby available for crosslinking alginate. Therefore, we hypothesized that compounding poly(vinyl alcohol) with Ca salts of lower solubility would minimize intra-composite crosslinking.

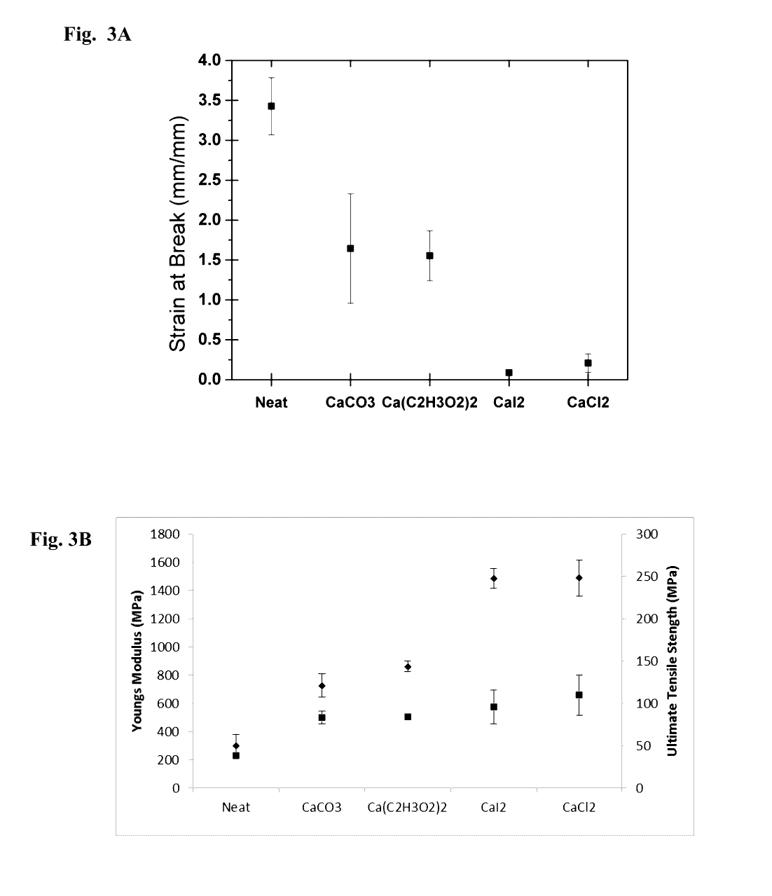

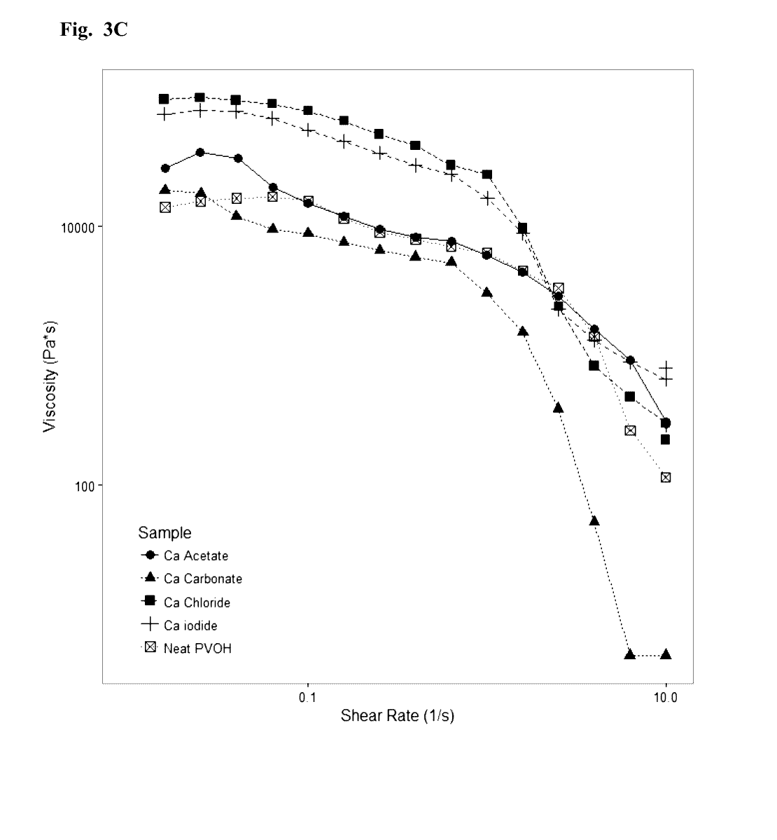

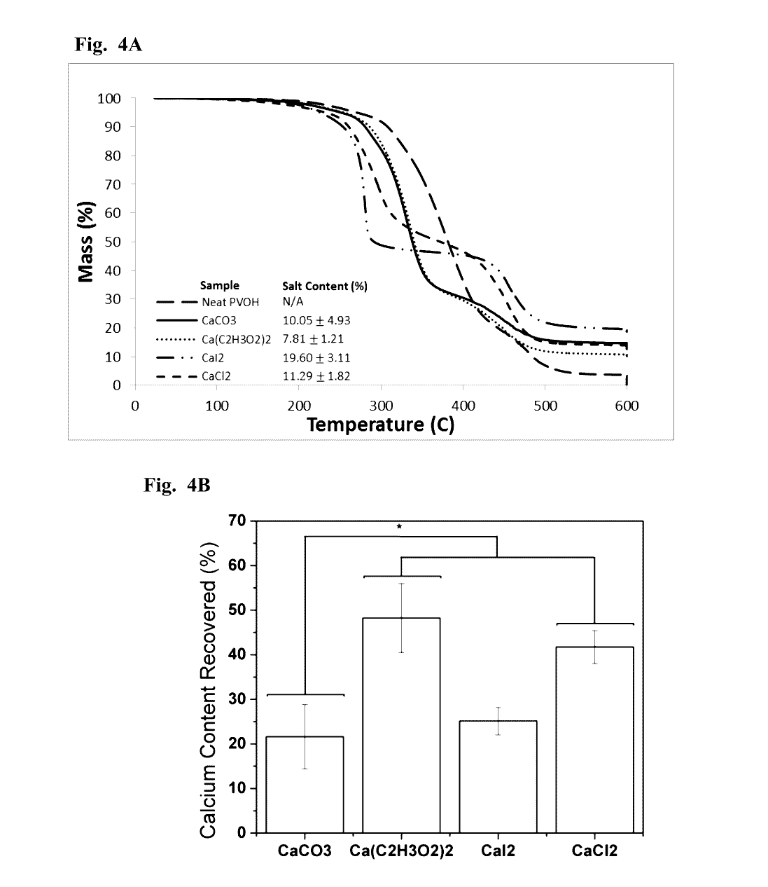

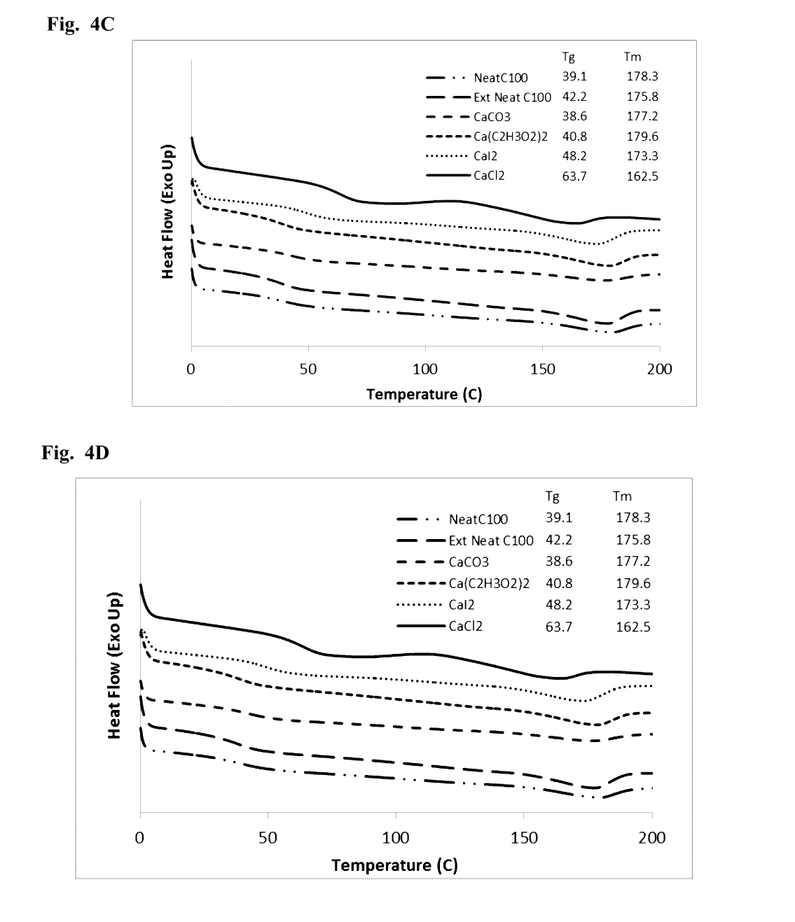

To test this theory, poly(vinyl alcohol) was also compounded with calcium iodide (CaI₂), calcium acetate (Ca(C₂H₃O₂)₂), or calcium carbonate (CaCO₃) salts, listed in order of decreasing solubility (Supplemental Table 2). Then, each composites' mechanical properties and calcium release were analyzed in detail (FIGS. 3 and 4). Tensile strength tests on injection molded, poly(vinyl alcohol)-Ca, ASTM D638 Type V specimens showed that the compounded calcium salts decrease composite ductility, i.e. strain at break (24) and increase modulus and ultimate tensile strength (UTS) compared to neat poly(vinyl alcohol) (FIGS. 3A and B). The Ca salts' effect on composite modulus and UTS is directly correlated with their solubility while the effect on composite ductility is in inversely correlated. These results agree with traditional polymer filler theory taking into account polymer chain crosslinking by solvated Ca²⁺ ions. Notably, sacrificial templates produced from poly(vinyl alcohol)-Ca(C₂H₃O₂)₂ or CaCO₃ composites versus carbohydrate glass would have enhanced handling durability due to a 50-fold increase in ductility(13).

Rheological measurements were conducted to assess each poly(vinyl alcohol)-Ca composite's viscosity and thereby relative ease of manufacturing by extrusion and injection molding. Using a parallel plate rheometer to measure viscosity at continuous shear rates, poly(vinyl alcohol)-CaI₂ or —CaCl₂ composites displayed a viscosity ˜10 fold higher than poly(vinyl alcohol)-neat, —Ca(C₂H₃O₂)₂, or —CaCO₃ across an order of magnitude shear rate range (0.1-1 per sec) (FIG. 3C). Similar to modulus and UTS data, the composite's viscosity was directly correlated to Ca salt solubility. The decreased viscosity of poly(vinyl alcohol)-neat, —Ca(C₂H₃O₂)₂, or —CaCO₃ composites was observed to facilitate extrusion and injection molding processes as well as minimized noticeable polymer degradation compared to poly(vinyl alcohol)-CaI₂ or —CaCl₂. As discussed later, optimizing the poly(vinyl alcohol)-Ca composite's viscosity was critical for feasible injection molding of sacrificial templates with microscale dimensions, i.e. micro-injection molding.

### 3.3 Optimizing Calcium Release Properties of Poly(Vinyl Alcohol)-Ca Templates

The lower solubility of calcium acetate and carbonate salts made them ideal compounding agents for producing poly(vinyl alcohol)-Ca composites with optimal mechanical properties. However, it remained unknown how their decreased solubility would affect Ca²⁺ ion release upon composite dissolution. To calculate this quantity, we first needed to know the actual weight percent of calcium salt in each poly(vinyl alcohol) composite. Since poly(vinyl alcohol) but not the compounded calcium salts decomposes below 600° C., thermogravimetric analysis (TGA), which measures mass loss over increasing temperatures, was executed on poly(vinyl alcohol) compounded with ˜10% (w/w) calcium salts. As shown in FIG. 4A, the compounded salts persisted in the ash content at 600° C. When normalized to the remaining neat poly(vinyl alcohol) ash, the persistent salt masses were found to be consistent with compounded values except for the poly(vinyl alcohol)-CaI₂ composite. Considerable poly(vinyl alcohol) degradation was observed during poly(vinyl alcohol)-CaI₂ extrusion, and this skewed the TGA results since degraded poly(vinyl alcohol) does not burn off. Also, the onset of degradation (first curve inflection point) of all poly(vinyl alcohol)-Ca composites occurred at lower temperatures than the neat composite, potentially indicating disruption of poly(vinyl alcohol) crystallinity by Ca²⁺ ions. This further corroborates the presence of intra-composite poly(vinyl alcohol)-Ca crosslinking.

A complexometric calcium titration was performed on the poly(vinyl alcohol)-Ca composites to assess the amount of Ca²⁺ ions released upon dissolution in deionized water (FIG. 4B). Calculating from the titration and TGA data, the average percent of released Ca²⁺ content was determined to never be greater than 50%. Also, the Ca²⁺ released from poly(vinyl alcohol)-Ca(C₂H₃O₂)₂ or —CaCl₂ composites was equivalent and significantly higher than that released from poly(vinyl alcohol)-CaI₂ and —CaCO₃. The lower Ca²⁺ content release of poly(vinyl alcohol)-CaI₂ composites could be due to polymer degradation during extrusion and the skewed TGA data whereas that of the poly(vinyl alcohol)-CaCO₃ is likely due to the salt's low solubility in water (Supplemental Table 2).

As a final analysis to explain <50% calcium recovery from poly(vinyl alcohol)-Ca(C₂H₃O₂)₂ or —CaCl₂ composites, we performed differential scanning calorimetry (DSC). This analytical technique measures the thermal energy input required to induce a 1° C. temperature change in the sample, thereby allowing characterization of the composite's glass transition (T_{g}) and melting (Tₘ) temperatures and crystallization enthalpy (ΔH_{c}). In DSC second heating curves (FIG. 4C), the T_{g} is indicated by the curve's first inflection point, and the Tₘ is indicated by the curve's last inflection point. We observed a direct correlation between the composites' T_{g} and an inverse correlation between the composites' Tₘ relative to its calcium salt's solubility (Supplemental Table 2), respectively. In the DSC cooling curve (FIG. 4D), the ΔH_{c} is calculated as the area under the curves' exothermic peak. In analyzing this material property, we observed an inverse correlation between the composites' ΔH_{c} and the compounded calcium salt's solubility. Moreover, the presence of a double exothermic peak in the neat poly(vinyl alcohol) and extruded neat poly(vinyl alcohol) samples versus the single peak in the extruded poly(vinyl alcohol)-Ca samples indicates that less types of crystals are being formed in salt containing samples. Collectively, the DSC results strongly suggest that the total compounded calcium content is not released from the poly(vinyl alcohol)-Ca composites upon dissolution due to intra-composite, poly(vinyl alcohol) polymer/Ca²⁺ crosslinking. Importantly, our detailed analysis of each composites mechanical and calcium release properties clearly imply that poly(vinyl alcohol)/Ca(C₂H₃O₂)₂ templates would provide superior sacrificial molding capabilities, handling durability, and micro-injection molding feasibility.

3.4 Micro-Injection Molded Poly(Vinyl Alcohol)-Ca(C₂H₃O₂)₂ Templates

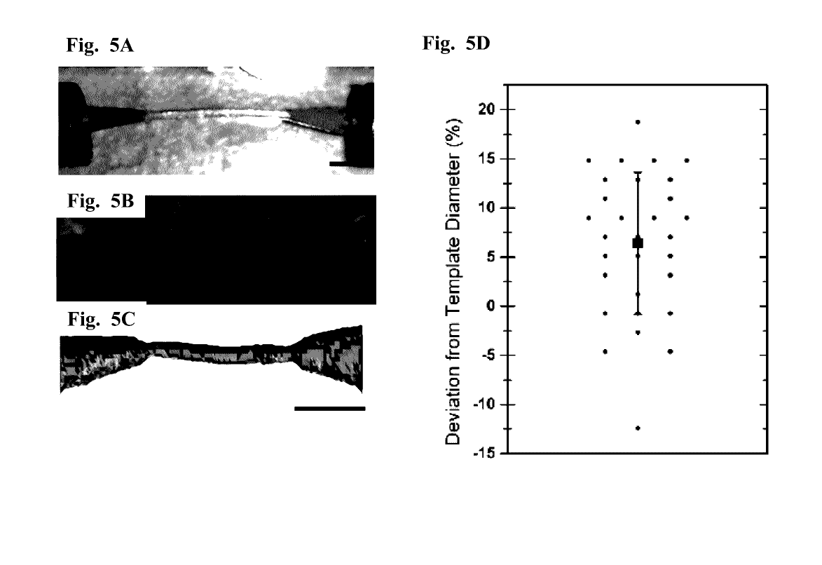

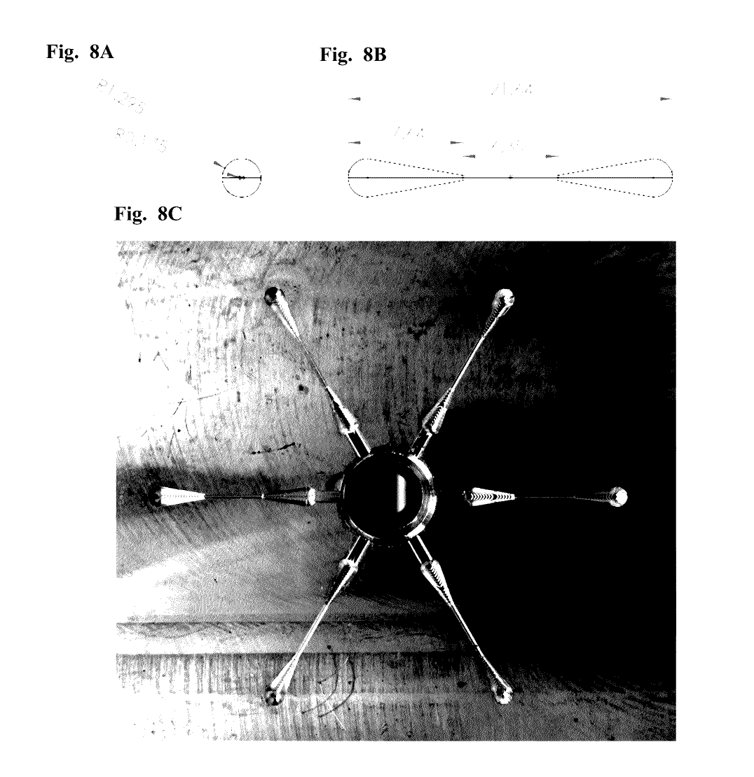

A microinjection mold was CNC-milled for a fiber template geometry 2.2 cm in length that immediately tapered from a 3 mm inlet/outlet diameter to a main fiber diameter of 500 μm (FIGS. 5A and 8). poly(vinyl alcohol)-Ca(C₂H₃O₂)₂ templates of 10% salt loading were successfully microinjection molded, but fabrication of similarly dimensioned and salt loaded poly(vinyl alcohol)-CaCl₂ templates failed. Post sacrificial molding of alginate hydrogels using the poly(vinyl alcohol)-Ca(C₂H₃O₂)₂ templates, the resulting micro-channels were filed with pigmented oil for photographing, and they were quantitatively analyzed by micro-CT imaging and reconstruction analysis (FIG. 5A). Dimensional analysis of the CT reconstructions revealed that the casted micro-channel diameter deviated from the template geometry by an average of 6.4±7.2% at any point along its long axis, i.e. 10 measurements per casted micro-channel (FIG. 5B). This demonstrates repeatable and precise casting of microscale architecture within diffusion limited, ionically crosslinked alginate hydrogels. Additionally, similarly dimensioned neat poly(vinyl alcohol) templates left no discernable micro-channel within alginate monoliths (data not shown).

### 3.5 Scalable 3-D Molding of Hydrogel Architecture

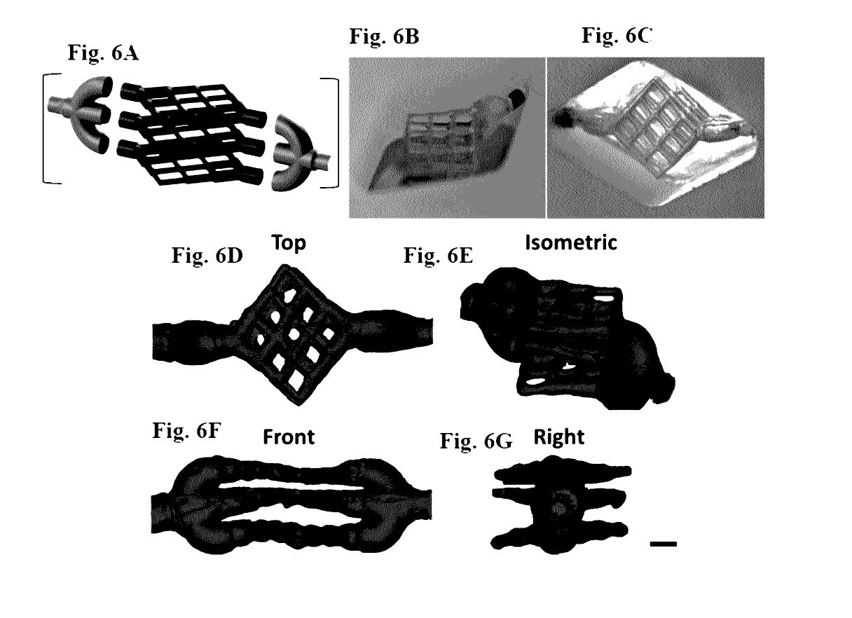

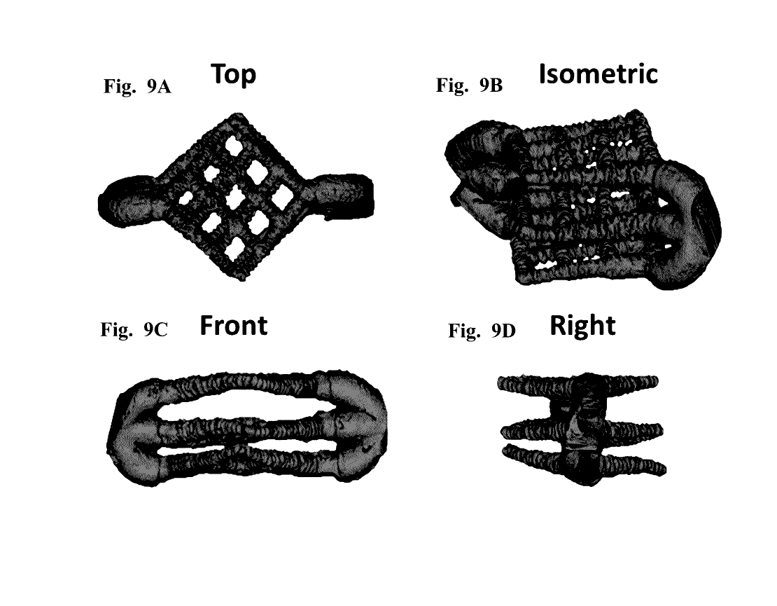

To explore the 3D scalability and versatility of injection molded poly(vinyl alcohol)-Ca(C₂H₃O₂)₂ templates, a set of CNC-milled, aluminum molds were fabricated to facilitate assembly of a 3D sacrificial template. The design allowed for freeform configuration of two modular components, a manifold and the previous lattice geometry, which connect via interference fit (FIG. 6A). After injection molding, 3D poly(vinyl alcohol)-Ca(C₂H₃O₂)₂ templates were assembled like Legos® (FIG. 6B), and used to sacrificially mold internal channel networks within polyacrylamide (FIG. 9) and alginate hydrogels (FIG. 6C). After template dissolution, the hydrogels were CT scanned and image reconstruction verified the resulting 3D channel networks patency and continuity (FIG. 6D). Thus, the design and injection molding of modular, poly(vinyl alcohol)-Ca(C₂H₃O₂)₂ components that can be assembled into 3D sacrificial templates represents a scalable and potentially limitless approach to precisely customize the architecture of bulk curing and ionically crosslinked hydrogels.

### 2. Conclusions

The ability to engineer tissue constructs with biomimetic morphologies and cytoarchitectures has been greatly enhanced by the development of techniques to fabricate biomaterial scaffolds with macro-to-microscale features. Sacrificial molding is a promising and scalable technique for rapidly casting complex architectures within hydrogel scaffolds. Yet, prior studies primarily demonstrated its efficacy using bulk curing hydrogels while noticeably less molding feasibility and precision is observed when applied to diffusion limited, ionically crosslinked hydrogels such as alginate. Furthermore, these studies performed limited to no quantitative analysis of their sacrificial molding approach's feature casting fidelity.

Here, we fabricated injection molded, poly(vinyl alcohol)-Ca templates to facilitate and optimize sacrificial molding within both bulk curing and ionically crosslinked hydrogels. Injection molded poly(vinyl alcohol) templates effectively mold bulk curing hydrogels such as polyacrylamide and PEG. However, the addition of calcium salts to the sacrificial templates was both necessary and sufficient to enable precise casting of microscale features within alginate monoliths. Extensive characterization unveiled that the poly(vinyl alcohol)-Ca composite's mechanical, material, and calcium release properties could be tuned based on the compounded calcium salt's solubility. Also, this analysis proved that poly(vinyl alcohol)-Ca(C₂H₃O₂)₂ possessed superior manufacturability and sacrificial template material properties, which were further demonstrated by precise casting of microscale 2D and modular 3D alginate hydrogel architectures. The micro-injection molded, poly(vinyl alcohol)-Ca(C₂H₃O₂)₂ templates described herein were developed to maximize production scalability, template design flexibility, and ease of implementation for researchers. We envision this will facilitate the manufacture of poly(vinyl alcohol)-Ca(C₂H₃O₂)₂ templates as a library of modular geometric pieces that can be assembled in Lego®-like configurations for rapid and precise customization of hydrogel scaffold architecture in diverse tissue engineering applications.

### REFERENCES

1. Ikada, Y., Challenges in tissue engineering. Journal of The Royal Society Interface, 2006. 3(10): p. 589-601.
2. Sun, J. and H. Tan, Alginate-based biomaterials for regenerative medicine applications. Materials, 2013. 6(4): p. 1285-1309.
3. Vacanti, J. P. and R. Langer, Tissue engineering: the design and fabrication of living replacement devices for surgical reconstruction and transplantation. The Lancet, 1999. 354: p. S32-S34.
4. Cao, Y., et al., Transplantation of Chondrocytes Utilizing a Polymer-Cell Construct to Produce Tissue Engineered Cartilage in the Shape of a Human Ear. Plastic and Reconstructive Surgery, 1997. 100(2): p. 297-302.
5. Murphy, S. V. and A. Atala, 3D bioprinting of tissues and organs. Nat Biotech, 2014. 32(8): p. 773-785.
6. Fernandez, J. G. and A. Khademhosseini, Micro-Masonry: Construction of 3D Structures by Microscale Self-Assembly. Advanced Materials, 2010. 22(23): p. 2538-2541.
7. Karande, T. S., J. L. Ong, and C. M. Agrawal, Diffusion in musculoskeletal tissue engineering scaffolds: design issues related to porosity, permeability, architecture, and nutrient mixing. Annals of biomedical engineering, 2004. 32(12): p. 1728-1743.
8. Mikos, A. G. and J. S. Temenoff, Formation of highly porous biodegradable scaffolds for tissue engineering. Electronic Journal of Biotechnology, 2000. 3(2): p. 23-24.
9. Nerem, R. M., The challenge of imitating nature. Principles of tissue engineering, 2000: p. 9-15.
10. Kolesky, D. B., et al., 3D Bioprinting of Vascularized, Heterogeneous Cell-Laden Tissue Constructs. Advanced Materials, 2014.
11. Bellan, L. M., et al., Fabrication of an artificial 3-dimensional vascular network using sacrificial sugar structures. Soft Matter, 2009. 5(7): p. 1354-1357.
12. Wu, W., et al., Direct-write assembly of biomimetic microvascular networks for efficient fluid transport. Soft Matter, 2010. 6(4): p. 739-742.
13. Miller, J. S., et al., Rapid casting of patterned vascular networks for perfusable engineered three-dimensional tissues. Nature Materials, 2012.
14. Tocchio, A., et al., Versatile fabrication of vascularizable scaffolds for large tissue engineering in bioreactor. Biomaterials, 2015. 45: p. 124-131.
15. Wang, X.-Y., et al., Engineering interconnected 3D vascular networks in hydrogels using molded sodium alginate lattice as the sacrificial template. Lab on a Chip, 2014. 14(15): p. 2709-2716.
16. Golden, A. P. and J. Tien, Fabrication of microfluidic hydrogels using molded gelatin as a sacrificial element. Lab Chip, 2007. 7(6): p. 720-725.
17. Cuchiara, M. P., et al., Multilayer microfluidic PEGDA hydrogels. Biomaterials, 2010. 31(21): p. 5491-5497.
18. Ashton, R. S., et al., Scaffolds based on degradable alginate hydrogels and poly(lactide-co-glycolide) microspheres for stem cell culture. Biomaterials, 2007. 28(36): p. 5518-5525.
19. Banerjee, A., et al., The influence of hydrogel modulus on the proliferation and differentiation of encapsulated neural stem cells. Biomaterials, 2009. 30(27): p. 4695-4699.
20. Rowley, J. A., G. Madlambayan, and D. J. Mooney, Alginate hydrogels as synthetic extracellular matrix materials. Biomaterials, 1999. 20(1): p. 45-53.
21. Paul, W. and C. P. Sharma, Chitosan and alginate wound dressings: a short review. Trends Biomater Artif Organs, 2004. 18(1): p. 18-23.
22. Osswald, T. A., L.-S. Turng, and P. J. Gramann, Injection molding handbook. 2008: Hanser Verlag.
23. Bonapasta, A. A., et al., Cross-linking of poly (vinyl alcohol) chains by Ca ions in macro-defect-free cements. Chemistry of materials, 2002. 14(3): p. 1016-1022.
24. Standard, A., D638: Standard Test Method for Tensile Properties of Plastics. West Conshohocken (PA): ASTM International, 2010.

### Supplemental

SUPPLEMENTAL TABLE 1.
Twin screw extrusion and microinjection molding
processing parameters.
Twin Screw Extruder Processing Parameters

| | |
|---|---|
| Zone 1 (° C.) | 160 |
| Zone 2 (° C.) | 165 |
| Zone 3 (° C.) | 170 |
| Zone 4 (° C.) | 173 |
| Zone 5 (° C.) | 175 |
| Zone 6 (° C.) | 177 |
| Zone 7 (° C.) | 178 |
| Zone 8 (° C.) | 180 |
| Load (%) | <40 |
| Screw Speed (rpm) | 50-70 |

Injection Molding Process Parameters

| | |
|---|---|
| Mold temperature (° C.) | 46 |
| Barrel temperature (° C.) | 190-200 |
| Injection pressure (bar) | 1250-1400 |
| Holding pressure (bar) | 1000 |
| Injection speed (cm³/s) | 55 |
| Holding time (s) | 2 |
| Cooling time (s) | 20 |

SUPPLEMENTAL TABLE 2
IUPAC-NIST Solubility Table (g/100 cc water)

| | | Temperature (° C.) | | | | |
|---|---|---|---|---|---|---|
| Compound | Formula | 0 | 10 | 20 | 30 | 40 |
| Calcium Carbonate | CaCO₃ | 0 | 0 | 0 | 0 | 0 |
| Calcium Sulfate | CaSO₄ | 0.2 | 0.2 | 0.3 | 0.3 | 0.3 |
| Calcium Acetate | Ca(C₂H₃O₂)₂ | 37.4 | 36 | 34.7 | 33.8 | 33.2 |
| Calcium Iodide | CaI₂ | 64.6 | 65.3 | 66 | 67.6 | 70.8 |
| Calcium Chloride | CaCl₂ | 59.5 | 64.7 | 74.5 | 100 | 128 |

The invention has been described in connection with what are presently considered to be the most practical and preferred embodiments. However, the present invention has been presented by way of illustration and is not intended to be limited to the disclosed embodiments. Accordingly, those skilled in the art will realize that the invention is intended to encompass modifications and alternative arrangements within the spirit and scope of the invention as set forth in the appended claims.

What is claimed is:

1. A method for controlled fabrication of internal spaces in a biocompatible hydrogel comprising:
(i) providing a sacrificial template that has a predefined shape and is immobilized within a casting chamber, wherein the sacrificial template comprises a water-soluble thermoplastic-divalent cationic salt composite material; and
(ii) introducing into the casting chamber a volume of hydrogel polymer solution comprising a hydrogel polymer that is cross-linkable by divalent cations, wherein the volume is sufficient to surround the sacrificial template;
whereby, (a) divalent cations diffuse out of the sacrificial template and cross-link the hydrogel polymer in the solution contacting the sacrificial template to form a hydrogel shell conforming to the shape of the sacrificial template; and (b) subsequent to formation of the hydrogel shell, the sacrificial template dissolves to form one or more internal spaces corresponding to the predefined shape.

2. The method of claim 1, wherein the water-soluble thermoplastic in the water-soluble thermoplastic-divalent cation composite material is selected from among poly(vinyl alcohol), poly(ethylene-oxide), poly(ethylene glycol), poly(lactic acid), poly(glycolic acid), and combinations thereof.

3. The method of claim 2, wherein water-soluble thermoplastic is poly(vinyl alcohol).

4. The method of claim 3, wherein the poly(vinyl alcohol)-divalent cationic composite material comprises calcium cations, magnesium cations, or barium cations.

5. The method of claim 4, wherein the poly(vinyl alcohol)-divalent cationic composite material comprises calcium cations.

6. The method of claim 5, wherein the poly(vinyl alcohol)-divalent cationic composite material comprises a calcium salt having a solubility of at least 0.01 g/ml to about 10 g/ml in water at 30° C.

7. The method of claim 5, wherein the poly(vinyl alcohol)-divalent composite cationic material comprises a calcium salt selected from the group consisting of: calcium acetate, calcium selenate, and calcium formate.

8. The method of claim 7, wherein the poly(vinyl alcohol)-divalent cationic composite material comprises calcium acetate.

9. The method of claim 8, wherein the poly(vinyl alcohol)-divalent cationic composite material comprises about 5% to about 30% calcium acetate.

10. The method of claim 1, wherein the hydrogel polymer solution comprises a divalent cation cross-linkable polymer selected from the group consisting of alginate, polysaccharides, xanthan gums, natural gum, agar, agarose, carrageenan, fucoidan, furcellaran, laminaran, hypnea, eucheuma, gum arabic, gum ghatti, gum karaya, gum tragacanth, locust beam gum, arabinogalactan, pectin, amylopectin, and ribo- or deoxyribonucleic acids.

11. The method of claim 10, wherein the hydrogel polymer solution comprises alginate.

12. The method of claim 1, wherein the shape of the one or more internal spaces deviates by no more than about 10% to about 15% from the predefined shape.

13. The method of claim 1, further comprising introducing into the casting chamber a cross-linking solution comprising divalent cations at a concentration sufficient to crosslink the hydrogel polymer, whereby remaining free hydrogel polymer in the volume of solution is cross-linked to form a hydrogel monolith comprising the one or more internal spaces.

14. The method of claim 1, wherein the hydrogel polymer solution further comprises a divalent cation at a concentration sufficient to partially cross-link the hydrogel polymer.

15. A sacrificial template comprising a water-soluble thermoplastic-divalent cationic salt composite material comprising a divalent cationic salt in an amount sufficient to initiate cross-linking of a divalent cation cross-linkable hydrogel polymer.

16. The sacrificial template of claim 15, wherein the water-soluble thermoplastic-divalent cation composite material is a poly(vinyl alcohol)-divalent cation composite material, and wherein the amount of the divalent cationic salt is insufficient to cause excessive cross-linking of the poly(vinyl alcohol).

17. The sacrificial template of claim 16, wherein the poly(vinyl alcohol)-divalent cation composite material is a poly(vinyl alcohol)-calcium composite material.

18. The sacrificial template of claim 17, wherein the poly(vinyl alcohol)-calcium composite material comprises calcium acetate.

19. The sacrificial template of claim 15, wherein the sacrificial template is fabricated by injection molding of the water-soluble thermoplastic-divalent cation composite material.

20. A kit comprising the sacrificial template of claim 15 and a divalent cation cross-linkable hydrogel polymer.
